(19) United States
(12) Patent Application Publication
(10) Pub. No.: US 2025/0122151 A1
(43) Pub. Date: Apr. 17, 2025

(54) METHOD FOR PRODUCING ORGANIC SULFONIC ACID COMPOUND

(71) Applicant: TAKEMOTO OIL & FAT CO., LTD., Aichi (JP)

(72) Inventor: Shun ISHIHARA, Aichi (JP)

(21) Appl. No.: 18/910,086

(22) Filed: Oct. 9, 2024

(30) Foreign Application Priority Data
Oct. 17, 2023 (JP) 2023-178683

(51) Int. Cl. C07C 303/22; C07C 303/44

(52) U.S. Cl. CPC C07C 303/22; C07C 303/44

(57) ABSTRACT

Provided is a method for producing an organic sulfonic acid compound, the method including a reaction step 1, a contact step 2, and a contact step 3, in which in the reaction step 1, an organic sulfonic acid compound is synthesized using a reaction base agent containing a chlorine-based compound; in the contact step 2, a solution of the organic sulfonic acid compound obtained in the reaction step 1 is contacted with an anion exchange resin, in the contact step 3, the solution of the organic sulfonic acid compound undergoing the contact step 2 is contacted with a cation exchange resin, and the anion exchange resin in the contact step 2 and the cation exchange resin in the contact step 3 are separated from each other.

### CROSS-REFERENCE TO RELATED APPLICATION

This application is based on Japanese Patent Application No. 2023-178683 filed with the Japan Patent Office on Oct. 17, 2023, the entire content of which is hereby incorporated by reference.

### BACKGROUND

### 1. Technical Field

The present invention relates to a method for producing an organic sulfonic acid compound.

### 2. Related Art

In technical fields of manufacturing and precision processing of devices such as electronic components or semiconductors, high-performance cleaning agents containing surfactants are used. Examples of contaminants or deposits that may occur in a manufacturing process of silicon wafers or semiconductors include dirt or dust called particles and various impurity ions. The particles on the semiconductors can cause malfunctions such as circuit breaks. As a result, the semiconductors cannot be used. This reduces product yield. Further, the impurity ions such as chlorine ions or metal ions on metal wiring of the semiconductors can enter gaps between the metal wiring. In this case, the semiconductors may not operate normally due to current leakage caused by the impurity ions. The presence of the impurity ions can present serious problems with manufactured devices, such as insufficient performance due to fluctuations in electrical characteristics. Furthermore, the chlorine ions on the metal wiring of the semiconductors tend to accelerate corrosion of the metal wiring. As a result, the metal wiring is easily damaged. This causes a resistance value of the metal wiring to increase. In this way, the presence of the particles or the impurity ions significantly affects quality of the semiconductors. In recent years, there has been a remarkable reduction in size of devices such as smartphones and personal computers. The semiconductors used in these devices are also becoming smaller. A line width of circuits in these miniaturized semiconductors is extremely small. As a result, smaller particles can cause deformation of the circuits, or the like. This requires that the particles and the impurity ions be removed with greater precision. Therefore, there is a demand for highly purified surfactants in which concentrations of the chlorine ions and the metal ions are further reduced.

With regard to a purification method of the surfactants, in addition to general methods using concentration, crystallization, or extraction, for example, a method using an ion exchange method using an ion exchange resin is known. For example, in a method disclosed in JP-A-2009-143842 (Patent Literature 1), an alkali metal salt of an organic sulfonic acid is subjected to an ion exchange method using a strongly acidic cation exchange resin. By treating an organic sulfonate in this manner, concentrations of various metal ions are reduced. Further, in a method disclosed in JP-A-2015-038051 (Patent Literature 2), an alkali metal salt and/or ammonium salt of an organic sulfonic acid is subjected to the ion exchange method using the strongly acidic cation exchange resin. By treating the organic sulfonate in this manner, the concentrations of various metal ions are reduced. Furthermore, in a method disclosed in JP-A-2001-064249 (Patent Literature 3), an aqueous solution of an alkane sulfonic acid and a basic anion exchange resin are brought into contact with each other in order to reduce a content rate of sulfuric acid in the alkane sulfonic acid.

However, when impurities are reduced by general purification methods such as crystallization and extraction, quality of the surfactant is far from quality required in the semiconductor field. Further, according to the method disclosed in Patent Literature 1 or Patent Literature 2, it is possible to reduce the concentration of the metal ions contained in a surfactant solution. However, it is unclear whether this method can reduce the concentration of the chlorine ions. According to the method disclosed in Patent Literature 3, it is possible to reduce concentration of sulfate ions contained in the surfactant solution. However, it is unclear whether this method can reduce the concentration of the chlorine ions or the concentration of the metal ions. None of the patent literatures explicitly states that the chlorine ions, the metal ions, and the like are reduced to quality required in the semiconductor manufacturing field.

The surfactant solution containing the organic sulfonic acid compound is used in the manufacture of semiconductors, for example, for the purpose of dispersion, surface modification, or cleaning. As methods for synthesizing the organic sulfonic acid compound, a manufacturing method using a chlorine-based compound and a manufacturing method using a sulfuric acid-based compound are widely known. In the manufacturing method using the chlorine-based compound, the synthesized organic sulfonic acid contains a large amount of chlorine ions derived from reactants such as sulfonic acid chloride. In the manufacturing method using the sulfuric acid-based compound, a chlorine-based solvent such as chloroform is often used as a solvent for reaction. Therefore, the synthesized sulfonic acid compound contains the chlorine ions. As described above, in the manufacturing method using the chlorine-based compound, the chlorine ions derived from the reactants such as the sulfonic acid chloride are contained in the organic sulfonic acid compound. Further, even in the manufacturing method using the sulfuric acid-based compound, when sodium salts and the like are formed, a large amount of chlorine ions derived from impurities in salts are contained in the organic sulfonic acid compound. Therefore, these chlorine ions are a problem in the semiconductor manufacturing field. In this situation, it is desired to reduce these chlorine ions.

Therefore, the present disclosure provides a method for producing an organic sulfonic acid compound, which can efficiently reduce chlorine ion content and metal ion content.

### SUMMARY

A method for producing an organic sulfonic acid compound, according to an embodiment of the present disclosure includes a reaction step 1, a contact step 2, and a contact step 3, in which in the reaction step 1, an organic sulfonic acid compound is synthesized using a reaction base agent containing a chlorine-based compound; in the contact step 2, a solution of the organic sulfonic acid compound obtained in the reaction step 1 is contacted with an anion exchange resin; in the contact step 3, the solution of the organic sulfonic acid compound undergoing the contact step 2 is contacted with a cation exchange resin; and the anion exchange resin in the contact step 2 and the cation exchange resin in the contact step 3 are separated from each other.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details.

In order to solve the above problems, the technique of the present disclosure includes means described below.
(1) A method for producing an organic sulfonic acid compound, the method including a reaction step 1, a contact step 2, and a contact step 3, in which in the reaction step 1, an organic sulfonic acid compound is synthesized using a reaction base agent containing a chlorine-based compound; in the contact step 2, a solution of the organic sulfonic acid compound obtained in the reaction step 1 is contacted with an anion exchange resin; in the contact step 3, the solution of the organic sulfonic acid compound undergoing the contact step 2 is contacted with a cation exchange resin; and the anion exchange resin in the contact step 2 and the cation exchange resin in the contact step 3 are separated from each other.
(2) The production method according to (1), in which the organic sulfonic acid compound is a surfactant.
(3) The production method according to (1), in which in the contact step 2, the solution having an organic sulfonic acid compound concentration of 1 to 50 mass % is contacted with the anion exchange resin.
(4) The production method according to (1), in which in the contact step 3, the solution having an organic sulfonic acid compound concentration of 1 to 40 mass % is contacted with the cation exchange resin.
(5) The production method according to (1), in which in the contact step 2, the solution is contacted with the anion exchange resin at a space velocity (an SV) of 0.3 to 4.9 h⁻¹.
(6) The production method according to (1), in which in the contact step 3, the solution is contacted with the cation exchange resin at a space velocity (an SV) of 0.3 to 4.9 h⁻¹.

Note that in description of the present embodiment, a numerical range indicated by “A to B” includes upper and lower limits unless otherwise specified. That is, “A to B” means “A or more and B or less.”

According to a production method of the present disclosure described above, it is possible to provide a method for producing an organic sulfonic acid compound, which can efficiently reduce chlorine ion content and metal ion content.

The method for producing the organic sulfonic acid compound according to the present embodiment includes a reaction step 1 of synthesizing the organic sulfonic acid compound, and a contact step 2 and a contact step 3 of performing ion exchange. Here, the ion exchange resin in the contact step 2 and the ion exchange resin in the contact step 3 are separated from each other.

### Reaction Step 1

In the reaction step 1, the organic sulfonic acid compound is synthesized using the reaction base agent containing the chlorine-based compound. In the present embodiment, the term “the reaction base agent containing the chlorine-based compound” refers to a series of reaction base agents contained in starting materials of reaction, such as a solvent or a catalyst. Examples of the reaction base agents include aromatic hydrocarbons such as benzene, octylbenzene, dodecylbenzene, and octadecylbenzene, and unsaturated hydrocarbons containing a double bond, such as octene, decene, undecene, dodecene, tetradecene, hexadecene, and octadecene. Of these, benzene, dodecylbenzene, octene, dodecene, hexadecene, and octadecene are preferred. One of these reaction base agents can be used alone. Alternatively, two or more reaction base agents may be used in combination.

### (Chlorine-Based Compound)

The chlorine-based compound used is not particularly limited. Examples of the chlorine-based compound include sodium chloride, potassium chloride, calcium chloride, magnesium chloride, ammonium chloride, aluminum chloride, hydrogen chloride, iron chloride, copper chloride, chloromethane, chloroethane, chlorosulfonic acid, chloroform, and chlorobenzene. One of these chlorine-based compounds can be used alone.

Alternatively, two or more chlorine-based compounds may be used in combination.

### (Organic Sulfonic Acid Compound)

A synthesis method of the reaction step 1 is not particularly limited. A conventionally known method can be used. Examples of the synthesis method include a method of sulfochlorination using the chlorine-based compound, and a method of neutralization with sodium hydroxide after a sulfonation reaction using a sulfuric acid-based compound or a sulfonic oxidation reaction using sulfur dioxide and oxygen. The organic sulfonic acid compound to be synthesized may be any of monosulfonic acid compounds, disulfonic acid compounds, and mixtures thereof. Further, alkyl groups contained in the organic sulfonic acid compound include linear alkyl groups and branched alkyl groups. There may be distribution in the number of carbon atoms in the alkyl group.

The organic sulfonic acid compound is preferably the surfactant. Examples of such an organic sulfonic acid compound include sodium octyl sulfonate, sodium dodecylbenzene sulfonate, and sodium octadecyl sulfonate. However, surfactants that can be used are not limited to these examples.

In the reaction step 1, a halogen (a compound containing chlorine) is produced as a by-product together with production of the organic sulfonic acid compound.

Alternatively, chlorine derived from the chlorine-based compound used as the solvent for the reaction is contained in the organic sulfonic acid compound. Therefore, in order to purify the synthesized organic sulfonic acid compound, the following contact steps 2 and 3 are carried out in this order.

### Contact Step 2

In contact step 2, a solution of the organic sulfonic acid compound obtained in reaction step 1 is contacted with an anion exchange resin. A purpose of the contact step 2 is mainly to allow the anion exchange resin to adsorb chlorine ions in the organic sulfonic acid compound. In the contact step 2 and the contact step 3 described below, a method of contacting the solution of the organic sulfonic acid compound with the ion exchange resin is not particularly limited. For example, a column method or a batch method may be used. From the viewpoint of work efficiency, the column method is preferred. A known device such as an ion exchange column can be used to contact with the ion exchange resin according to a method disclosed in the present disclosure.

The solution of the organic sulfonic acid compound obtained in the reaction step 1 is passed through the anion exchange resin. Then, the chlorine ions contained in the solution of the organic sulfonic acid compound are adsorbed by the anion exchange resin. As a result, chlorine ion concentration in the obtained purified liquid can be reduced. In the present disclosure, the contact step 2 is performed after the reaction step 1. That is, by performing anion exchange in the contact step 2, only the chlorine ions are selectively captured by the anion exchange resin from the organic sulfonic acid compound in a state synthesized in the reaction step 1. As a result, it is possible to effectively reduce the chlorine ion concentration. Note that the solution of the organic sulfonic acid compound is a solution obtained by dissolving the organic sulfonic acid compound synthesized in the reaction step 1 in a solvent suitable for the ion exchange method, such as ion exchange water.

### (Anion Exchange Resin)

The anion exchange resin used in the contact step 2 may be any of a strongly basic resin, a weakly basic resin, and a mixed resin thereof. Of these, the anion exchange resin containing a strongly basic anion exchange resin are preferred. The anion exchange resin used is not particularly limited. A known anion exchange resin can be used. A shape of the anion exchange resin is not limited to granular. The shape of the anion exchange resin may be powder, fiber, or film. A structure of the anion exchange resin may be a gel type or a macroporous type. One of these anion exchange resins can be used alone. Alternatively, two or more anion exchange resins may be used in combination.

A content ratio of the strongly basic anion exchange resin in the anion exchange resin is not particularly limited. A preferred content ratio is 50 vol % or more. When the content ratio of the strongly basic anion exchange resin is within this range, since the chlorine ions are adsorbed on the strongly basic anion exchange resin, the chlorine ion content in the solution of the organic sulfonic acid compound is effectively reduced.

The strongly basic anion exchange resin is an anion exchange resin into which a strongly basic functional group such as a quaternary ammonium base (R—N+R1R2R3) is introduced. Examples of the strongly basic anion exchange resin that can be used include type I strongly basic anion exchange resins such as Amberlite (registered trademark, the same below) IRA400J CI, IRA402BL, and IRA900J (all manufactured by DuPont, USA), Duolite (registered trademark, the same below) A113LF, A161JCL, and AGP (all manufactured by DuPont, USA), and Diaion (registered trademark, the same below) SA10A and SA11A (both manufactured by Mitsubishi Chemical Corporation). In addition, other examples thereof include type II strongly basic anion exchange resins such as Amberlite IRA410J, IRA910CT, and HPR4010 (all manufactured by DuPont, USA), Duolite A116 and A162LF (both manufactured by DuPont, USA), and Diaion SA20A and SA20ALL (both manufactured by Mitsubishi Chemical Corporation).

A weakly basic anion exchange resin is an anion exchange resin into which a weakly basic functional group such as a primary to tertiary amine is introduced. Examples of the weakly basic anion exchange resin include Amberlite IRA67, IRA96SB, and IRA98 (all manufactured by DuPont, USA), Duolite A368 MS, A378D, and A375LF (all manufactured by DuPont, USA), and Diaion WA10 and WA20 (both manufactured by Mitsubishi Chemical Corporation).

The anion exchange resin is preferably regenerated in advance to an OH type using a salt such as an aqueous solution of tetramethylammonium salt. The resulting OH type anion exchange resin is thoroughly washed, for example, with ion exchanged water or ultrapure water.

In the contact step 2, a concentration of the organic sulfonic acid compound in the solution of the organic sulfonic acid compound that is contacted with the anion exchange resin is not particularly limited. The concentration is preferably 1 to 50 mass %. When the concentration in the solution is too high, viscosity of the solution increases. Therefore, molecular motion in the solution is restricted. As a result, since the number of times the ion exchange resin is contacted with the chlorine ions decreases, the chlorine ion concentration is less likely to be efficiently reduced.

The concentration of the organic sulfonic acid compound in the solution of the organic sulfonic acid compound that is contacted with the anion exchange resin is more preferably 15 to 35 mass %. When the concentration is too low, an amount of water required for dilution increases. Therefore, a flow rate per unit time of the solution of the organic sulfonic acid compound when performing the ion exchange must be increased. Therefore, a too low concentration is not appropriate from the viewpoint of productivity.

The space velocity (SV) when the solution of the organic sulfonic acid compound is subjected to ion exchange treatment in the contact step 2 is preferably set to 0.3 to 4.9 h⁻¹, and more preferably 0.3 to 1.0 h⁻¹. When a speed at which the liquid is passed is too fast, it is difficult to sufficiently adsorb the chlorine ions. Note that a space velocity (SV) value, that is, an SV value is a unit that indicates how many times an amount of treated water is passed per hour relative to an amount of the ion exchange resin filled.

### Contact Step 3

In the contact step 3, the solution of the organic sulfonic acid compound undergoing the contact step 2 is contacted with the cation exchange resin. A purpose of the contact step 3 is mainly to allow the cation exchange resin to adsorb metal ions in the organic sulfonic acid compound. When the solution of the organic sulfonic acid compound undergoing the contact step 2 is passed through the cation exchange resin, the metal ions contained in the solution of the organic sulfonic acid compound are adsorbed by the cation exchange resin. As a result, not only the chlorine ions but also the metal ions in the resulting purified liquid can be reduced.

### (Cation Exchange Resin)

The cation exchange resin used in the contact step 3 may be any of a strongly acidic resin, a weakly acidic resin, and a mixed resin thereof. Of these, the cation exchange resin containing a strongly acidic cation exchange resin are preferred. The cation exchange resin used is not particularly limited. A known cation exchange resin can be used. A shape of the cation exchange resin is not limited to granular. The shape of the cation exchange resin may be powder, fiber, or film. A structure of the cation exchange resin may be a gel type or a macroporous type. One of these cation exchange resins can be used alone. Alternatively, two or more cation exchange resins may be used in combination.

A content ratio of the strongly acidic cation exchange resin in the cation exchange resin is not particularly limited. A preferred content ratio is 90 vol % or more. When the content ratio of the strongly acidic cation exchange resin is within this range, since the metal ions are adsorbed on the strongly acidic cation exchange resin, the metal ion content in the solution of the organic sulfonic acid compound is effectively reduced.

A strongly acidic cation exchange resin is a cation exchange resin that has a relatively large adsorption capacity for cationic components. The strongly acidic cation exchange resin has a strongly acidic exchange group such as a sulfonic acid group (R—SO3-H+) as a functional group. Examples of the strongly acidic cation exchange resin that can be used include Amberlite IR120BNa, IR-124Na, and 200CTNa (all manufactured by DuPont, USA), Duolite C20, C20LF, and C255LFH (all manufactured by DuPont, USA), and Diaion SK104H, SK110, and SK1B (all manufactured by Mitsubishi Chemical Corporation).

A weakly acidic cation exchange resin is a cation exchange resin that has a relatively small adsorption capacity for the cationic components. The weakly acidic cation exchange resin has a weakly acidic exchange group such as a carboxylic acid group (R—COO—H+) as the functional group. Examples of the weakly acidic cation exchange resin that can be used include Amberlite IRC76, FPC3500, and HPR8400 (all manufactured by DuPont, USA), Duolite C476 (manufactured by DuPont, USA), and Diaion WK10 and WK11 (both manufactured by Mitsubishi Chemical Corporation).

The cation exchange resin is preferably regenerated in advance to an H type using an acid such as hydrochloric acid or sulfuric acid. The resulting H type cation exchange resin is thoroughly washed, for example, with ion-exchanged water or ultrapure water.

In the contact step 3, a concentration of the organic sulfonic acid compound in the solution of the organic sulfonic acid compound that is contacted with the cation exchange resin is not particularly limited. The concentration is preferably 1 to 40 mass %. When the concentration in the solution is too high, viscosity of the solution increases. Therefore, molecular motion in the solution is restricted. As a result, since the number of times the ion exchange resin is contacted with the metal ions decreases, the metal ion concentration is less likely to be efficiently reduced.

The concentration of the organic sulfonic acid compound in the solution of the organic sulfonic acid compound that is contacted with the cation exchange resin is more preferably 15 to 30 mass %. When the concentration is too low, an amount of water required for dilution increases. Therefore, a flow rate per unit time of the solution of the organic sulfonic acid compound when performing the ion exchange must be increased. Therefore, a too low concentration is not appropriate from the viewpoint of productivity.

The space velocity (SV) when the solution of the organic sulfonic acid compound is subjected to ion exchange treatment in the contact step 3 is preferably set to 0.3 to 4.9⁻¹, and more preferably 0.3 to 1.0 h⁻¹. When the speed at which the liquid is passed is too fast, it is difficult to sufficiently adsorb the metal ions.

In the present embodiment, the reaction step 1, the contact step 2, and the contact step 3 are carried out in this order. Thus, the organic sulfonic acid compound is produced. In the reaction step 1, the organic sulfonic acid compound is synthesized using the reaction base agent containing the chlorine-based compound. In the contact step 2, by performing anion exchange with the anion exchange resin, only the chlorine ions are selectively captured by the anion exchange resin from the organic sulfonic acid compound in this synthesized state. Thereafter, in the contact step 3, by performing cation exchange with the cation exchange resin, the metal ions can be removed from the solution of the organic sulfonic acid compound undergoing the contact step 2. Therefore, as a result of purification, an organic sulfonic acid compound can be obtained in which the concentrations of the chlorine ions and the metal ions are reduced.

On the other hand, when the contact step 2 and the contact step 3 are carried out in a reverse order, the organic sulfonic acid compound synthesized in reaction step 1 using the reaction base agent containing the chlorine-based compound undergoes cation exchange in the contact step 3. Therefore, when the metal ions are adsorbed on the cation exchange resin in the cation exchange in the contact step 3, the organic sulfonic acid compound changes to an organic alkyl sulfonic acid. Thereafter, when the anion exchange is performed in the contact step 2, the chlorine ions are adsorbed on the anion exchange resin. At this time, the sulfonic acid is also adsorbed on the anion exchange resin.

Therefore, as a result of purification, an organic sulfonic acid compound from which the chlorine ions and the metal ions are removed cannot be obtained.

Furthermore, in the present embodiment, the anion exchange resin in the contact step 2 and the cation exchange resin in the contact step 3 are separated from each other. On the other hand, when the anion exchange resin and the cation exchange resin are in contact with each other, carbonate ions and protons are generated at a contact portion. That is, the anion exchange resin comes into contact with air and adsorbs carbon dioxide. Then, carbonate ions are liberated from the anion exchange resin. Then, the liberated carbonate ions come into contact with protons (H+) generated from the cation exchange resin. As a result, the carbon dioxide is generated as bubbles. This makes it difficult for the solution of the organic sulfonic acid compound to pass through the anion exchange resin. As a result, the ion exchange cannot be performed sufficiently. In addition, also when the contact step 2 and the contact step 3 are performed using a mixed resin of anion exchange resin and cation exchange resin, carbon dioxide bubbles are generated in the same way. As a result, the ion exchange cannot be performed sufficiently.

The production method described above makes it possible to obtain an organic sulfonic acid compound with reduced concentrations of the chlorine ions and the metal ions. Moreover, it is possible to avoid, for example, generation of the bubbles during liquid passage, which prevents ion exchange from being performed. As a result, an organic sulfonic acid compound with a high degree of purification can be efficiently produced.

### EXAMPLES

Operations and effects of the present embodiment will be described in more detail below with reference to Examples and Comparative Examples. However, the present embodiment is not limited to these Examples. In the following Examples and Comparative Examples, % means mass %. Table 1 shows compounds synthesized in the reaction step 1 and synthesis methods thereof in each of Examples and Comparative Examples. Further, Table 1 shows solution concentration, the SV value, and test method in each of the contact step 2 and the contact step 3.

### Reaction Step 1

In the reaction step 1, the following synthesis methods 1 to 3 were performed. In Examples 1 to 5, 7 to 10, and 14 to 31, and Comparative Examples 1 to 5, a synthesis method 1 was performed. In Example 6, a synthesis method 2 was performed. In Example 32, a synthesis method 3 was performed. In Examples 11 to 13, the synthesis method 1 and the synthesis method 3 were performed.

### Example 1

### Synthesis Method 1

116.5 g (1.0 mol, 1.0 eq.) of chlorosulfonic acid was added dropwise over 10 minutes to 246.4 g (1.0 mol) of dodecylbenzene weighed into a 500 mL four-neck flask. After addition, the mixture was heated to 150° C. Then, the reaction was carried out for 10 hours. The above reactants were added to 500 g of water weighed into a 2 L beaker. The reaction mixture was stirred at 70° C. for 2 hours and then allowed to cool. Subsequently, a 30 wt % sodium hydroxide aqueous solution was added until the mixture became neutral. In this way, the reaction product was recovered as a sodium dodecylbenzenesulfonate aqueous solution. Thereafter, the resulting aqueous solution was distilled under reduced pressure to remove water and unreacted dodecylbenzene. The solid obtained was extracted with chloroform, and from the obtained organic layer, the chloroform was distilled off under reduced pressure. In this way, sodium dodecylbenzenesulfonate from which impurities had been removed was obtained.

### Examples 2 to 5, 7 to 10, and 14 to 31, and Comparative Examples 1 to 5

Sulfonic acid compounds listed in Table 1 were obtained in the same manner as in the synthesis method 1, except that the reaction base agent in the synthesis method 1 was changed from dodecylbenzene to an unsaturated hydrocarbon (C8 to C18) containing a double bond.

### Example 6

### Synthesis Method 2

113.4 g (1.2 mol, 1.0 eq.) of 20% fuming sulfuric acid was added dropwise over 10 minutes to 295.7 g (1.2 mol) of dodecylbenzene weighed into a 500 mL four-neck flask. After addition, the mixture was heated to 160° C. Then, the reaction was carried out for 4 hours. The above reactants were added to 700 g of water weighed into a 2 L beaker. The reaction mixture was stirred at 70° C. for 2 hours and then allowed to cool. Subsequently, a 30 wt % sodium hydroxide aqueous solution was added until the mixture became neutral. In this way, the reaction product was recovered as a sodium dodecylbenzenesulfonate aqueous solution. Thereafter, the resulting aqueous solution was distilled under reduced pressure to remove water and unreacted dodecylbenzene. The solid obtained was extracted with chloroform, and from the obtained organic layer, the chloroform was distilled off under reduced pressure. In this way, sodium dodecylbenzenesulfonate from which impurities had been removed was obtained.

### Example 11

Sodium dodecylbenzenesulfonate was obtained by the synthesis method 1. Sodium dodecylbenzenedisulfonate was obtained by the synthesis method 3 below. The sulfonic acid compound of Example 11 was prepared at a ratio of sodium dodecylbenzenesulfonate/sodium dodecylbenzenedisulfonate=9:1.

### Synthesis Method 3

226.7 g (2.4 mol, 2.0 eq.) of 20% fuming sulfuric acid was added dropwise over 10 minutes to 295.7 g (1.2 mol) of dodecylbenzene weighed into a 500 mL four-neck flask. After addition, the mixture was heated to 160° C. Then, the reaction was carried out for 4 hours. The above reactants were added to 700 g of water weighed into a 2 L beaker. The reaction mixture was stirred at 70° C. for 2 hours and then allowed to cool. Subsequently, a 30 wt % sodium hydroxide aqueous solution was added until the mixture became neutral. In this way, the reaction product was recovered as a sodium dodecylbenzenedisulfonate aqueous solution. Thereafter, the resulting aqueous solution was distilled under reduced pressure to remove water and unreacted dodecylbenzene. The solid obtained was extracted with chloroform, and from the obtained organic layer, the chloroform was distilled off under reduced pressure. In this way, sodium dodecylbenzenedisulfonate from which impurities had been removed was obtained.

### Examples 12 and 13

Sodium alkyl (C10 to C18) sulfonate of Example 12 and sodium tetradecyl sulfonate of Example 13 were obtained in the same manner as in Synthesis Method 1, except that the reaction base agent in Synthesis Method 1 was changed from dodecylbenzene to an unsaturated hydrocarbon (C10 to C18) containing a double bond. Sodium alkyl (C10 to C18) disulfonate of Example 12 and tetradecyl disulfonic acid of Example 13 were obtained in the same manner as in the synthesis method 3, except that the reaction base agent in the synthesis method 3 was changed from dodecylbenzene to an unsaturated hydrocarbon (C10 to C18) containing a double bond.

The sulfonic acid compound of Example 12 was prepared at a ratio of sodium alkyl (C10 to C18) sulfonate:sodium alkyl (C10 to C18) disulfonate=1:9. The sulfonic acid compound of Example 13 was prepared at a ratio of sodium tetradecyl sulfonate:sodium tetradecyl disulfonate=1:1.

### Example 32

Sodium benzenedisulfonate was obtained in the same manner as in the synthesis method 3, except that the reaction base agent in the synthesis method 3 of Example 11 was changed from dodecylbenzene to benzene.

TABLE 1

| | Reaction step 1 | | Contact step 2 | | | Contact step 3 | | |
|---|---|---|---|---|---|---|---|---|
| | | Synthesis | Solution | SV | Test | Solution | SV | Test |
| Category | Synthetic compound | method | concentration (%) | value (h⁻¹) | method | concentration (%) | value (h⁻¹) | method |
| Example 1 | Sodium | 1 | 30 | 0.4 | 2-1 | 25 | 0.5 | 3-1 |
| | dodecylbenzenesulfonate | | | | | | | |
| Example 2 | Sodium | 1 | 30 | 0.4 | 2-1 | 24 | 0.6 | 3-1 |
| | octadecyl sulfonate | | | | | | | |
| Example 3 | Sodium | 1 | 35 | 0.5 | 2-1 | 25 | 0.8 | 3-1 |
| | dodecyl sulfonate | | | | | | | |
| Example 4 | Sodium | 1 | 32 | 0.5 | 2-1 | 24 | 0.8 | 3-1 |
| | octyl sulfonate | | | | | | | |
| Example 5 | Sodium | 1 | 30 | 0.6 | 2-1 | 25 | 0.5 | 3-1 |
| | hexadecyl sulfonate | | | | | | | |
| Example 6 | Sodium | 2 | 35 | 0.8 | 2-1 | 30 | 0.8 | 3-1 |
| | dodecylbenzenesulfonate | | | | | | | |
| Example 7 | Sodium | 1 | 32 | 0.3 | 2-1 | 25 | 0.7 | 3-1 |
| | alkyl (C10 to C18) sulfonate | | | | | | | |
| Example 8 | Sodium | 1 | 32 | 0.6 | 2-1 | 25 | 1.0 | 3-1 |
| | dodecylbenzenedisulfonate | | | | | | | |
| Example 9 | Sodium | 1 | 35 | 0.5 | 2-1 | 25 | 0.6 | 3-1 |
| | alkyl (C12 to C16) sulfonate | | | | | | | |
| Example 10 | Sodium | 1 | 32 | 0.5 | 2-1 | 24 | 0.5 | 3-1 |
| | alkyl (C13 to C18) sulfonate | | | | | | | |
| Example 11 | Sodium dodecylbenzenesulfonate/ | 1 and 3 | 30 | 0.5 | 2-1 | 24 | 0.8 | 3-1 |
| | Sodium | | | | | | | |
| | dodecylbenzenedisulfonate = 9:1 | | | | | | | |
| Example 12 | Sodium alkyl (C10 to C18) sulfonate/ | 1 and 3 | 35 | 0.8 | 2-1 | 30 | 0.8 | 3-1 |
| | Sodium alkyl (C10 to C18) | | | | | | | |
| | disulfonate = 1:9 | | | | | | | |
| Example 13 | Sodium tetradecyl sulfonate/ | 1 and 3 | 35 | 0.3 | 2-1 | 30 | 0.8 | 3-1 |
| | Sodium tetradecyl | | | | | | | |
| | disulfonate = 1:1 | | | | | | | |
| Example 14 | Sodium octyl sulfonate | 1 | 32 | 0.6 | 2-2 | 26 | 0.5 | 3-1 |
| Example 15 | Sodium hexadecyl sulfonate | 1 | 25 | 0.6 | 2-1 | 22 | 0.5 | 3-2 |
| Example 16 | Sodium tetradecyl sulfonate | 1 | 24 | 0.8 | 2-2 | 18 | 0.6 | 3-2 |
| Example 17 | Sodium octyl sulfonate | 1 | 18 | 0.6 | 2-1 | 8 | 0.8 | 3-1 |
| Example 18 | Sodium octyl sulfonate | 1 | 32 | 0.2 | 2-1 | 24 | 0.4 | 3-1 |
| Example 19 | Sodium octyl sulfonate | 1 | 35 | 0.4 | 2-1 | 30 | 0.2 | 3-1 |
| Example 20 | Sodium octyl sulfonate | 1 | 13 | 0.3 | 2-1 | 8 | 0.3 | 3-1 |
| Example 21 | Sodium octyl sulfonate | 1 | 32 | 0.4 | 2-1 | 25 | 2.0 | 3-1 |
| Example 22 | Sodium octyl sulfonate | 1 | 32 | 0.4 | 2-1 | 18 | 5.0 | 3-1 |
| Example 23 | Sodium octyl sulfonate | 1 | 40 | 0.4 | 2-1 | 25 | 0.4 | 3-1 |
| Example 24 | Sodium octyl sulfonate | 1 | 30 | 2.0 | 2-1 | 25 | 0.6 | 3-1 |
| Example 25 | Sodium octyl sulfonate | 1 | 18 | 5.0 | 2-1 | 16 | 0.6 | 3-1 |
| Example 26 | Sodium octyl sulfonate | 1 | 48 | 0.5 | 2-1 | 45 | 0.8 | 3-1 |
| Example 27 | Sodium octyl sulfonate | 1 | 60 | 0.3 | 2-1 | 25 | 0.5 | 3-1 |
| Example 28 | Sodium octyl sulfonate | 1 | 60 | 0.5 | 2-1 | 52 | 0.8 | 3-1 |
| Example 29 | Sodium octyl sulfonate | 1 | 35 | 0.6 | 2-3 | 25 | 0.6 | 3-1 |
| Example 30 | Sodium octyl sulfonate | 1 | 35 | 0.6 | 2-4 | 25 | 0.6 | 3-1 |
| Example 31 | Sodium octyl sulfonate | 1 | 35 | 0.6 | 2-1 | 25 | 0.6 | 3-3 |
| Example 32 | Sodium benzenedisulfonate | 3 | 35 | 0.3 | 2-1 | 30 | 0.4 | 3-1 |
| Comparative | Sodium octyl sulfonate | 1 | — | — | — | 35 | 0.5 | 3-1 |
| Example 1 | | | | | | | | |
| Comparative | Sodium octyl sulfonate | 1 | 25 | 0.3 | 3-1 | 20 | 0.4 | 2-1 |
| Example 2 | | | | | | | | |
| Comparative | Sodium octyl sulfonate | 1 | 25 | 0.3 | 2-1 | — | — | — |
| Example 3 | | | | | | | | |
| Comparative | Sodium ostyl sulfonate | 1 | 35 | 0.5 | 2-5 | — | — | — |
| Example 4 | | | | | | | | |
| Comparative | Sodium octyl sulfonate | 1 | 32 | 0.5 | 2-6 | — | — | — |
| Example 5 | | | | | | | | |

The sulfonic acid compound synthesized in the reaction step 1 was diluted with water to produce an aqueous solution of the organic sulfonic acid compound. Table 1 shows the concentration of the organic sulfonic acid compound in the resulting aqueous solution. Thereafter, the contact step 2 and the contact step 3 were carried out in this order.

### Contact Step 2

In the contact step 2, the following test methods 2-1 to 2-6 were performed. In Examples 1 to 13, 15, 17 to 28, and 31 to 32, and Comparative Example 3, a test method 2-1 was performed. In Examples 14 and 16, a test method 2-2 was performed. In Example 29, a test method 2-3 was performed. In Example 30, a test method 2-4 was performed. In Comparative Example 4, a test method 2-5 was performed. In Comparative Example 5, a test method 2-6 was performed. In Comparative Example 2, a test method 3-1 described later was performed.

### (Test Method 2-1)

A 300 ml column set vertically was filled with 200 ml of gel-type strongly basic anion exchange resin (trade name “Amberlite IRA400J Cl”). Here, the strongly basic anion exchange resin was regenerated to the OH type in advance using a IN tetramethylammonium hydroxide aqueous solution. The anion exchange resin was thoroughly washed with 4000 g of pure water and then left to stand for 24 hours. Subsequently, the pure water injected into the column was kept at a constant temperature within a range of 15 to 25° C. Thereafter, the aqueous solution of the organic sulfonic acid compound adjusted to the same temperature as the pure water in the column was passed through the column at the space velocity (SV) listed in Table 1.

### (Test Method 2-2)

The aqueous solution of the organic sulfonic acid compound was passed through the column at the space velocity (SV) listed in Table 1 in the same manner as in the test method 2-1, except that an MR-type strongly basic anion exchange resin (trade name “Amberlite IRA900J”) was used instead of the gel-type strongly basic anion exchange resin (trade name “Amberlite IRA400J Cl”) in the test method 2-1.

### (Test Method 2-3)

The aqueous solution of the organic sulfonic acid compound was passed through the column at the space velocity (SV) listed in Table 1 in the same manner as in the test method 2-1, except that a gel-type weakly basic anion exchange resin (trade name “Amberlite IRA67”) was used instead of the gel-type strongly basic anion exchange resin (trade name “Amberlite IRA400J Cl”) in the test method 2-1.

### (Test Method 2-4)

100 ml of the gel-type strongly basic anion exchange resin (trade name “Amberlite IRA400J Cl”) in the test method 2-1 and 100 ml of a gel-type weakly basic anion exchange resin (trade name “Amberlite IRA67”) in the test method 2-3 were mixed uniformly. A vertically set column with a capacity of 300 ml was filled with the above mixed resin. Subsequently, the pure water injected into the column was kept at a constant temperature within the range of 15 to 25° C. Thereafter, the aqueous solution of the organic sulfonic acid compound adjusted to the same temperature as the pure water in the column was passed through the column at the space velocity (SV) listed in Table 1.

### (Test Method 2-5)

100 ml of an MR type strongly acidic cation exchange resin (trade name “Amberlite 200CT”) was regenerated to H type using IN dilute hydrochloric acid. Further, 100 ml of the MR type strongly basic anion exchange resin (trade name “Amberlite IRA900J”) was regenerated to OH type using IN tetramethylammonium hydroxide aqueous solution. Thereafter, the regenerated cation exchange resin and anion exchange resin were mixed uniformly. A vertically set column with a capacity of 300 ml was filled with the above mixed resin. Subsequently, the pure water injected into the column was kept at a constant temperature within the range of 15 to 25° C. Thereafter, the aqueous solution of the organic sulfonic acid compound adjusted to the same temperature as the pure water in the column was passed through the column at the space velocity (SV) listed in Table 1.

### (Test Method 2-6)

A vertically set column with a capacity of 300 m was filled with 100 ml of the MR type strongly basic anion exchange resin (trade name “Amberlite IRA900J”). Subsequently, the column was filled with 100 ml of the MR type strongly acidic cation exchange resin (trade name “Amberlite 200CT”) that had been regenerated to H type in advance using IN dilute hydrochloric acid. At this time, the anion exchange resin and the cation exchange resin located above the anion exchange resin were in contact with each other inside the column. Subsequently, the pure water injected into the column was kept at a constant temperature within the range of 15 to 25° C. Thereafter, the aqueous solution of the organic sulfonic acid compound adjusted to the same temperature as the pure water in the column was passed through the column at the space velocity (SV) listed in Table 1.

### Contact Step 3

In the contact step 3, the following test methods 3-1 to 3-3 were performed. In Examples 1 to 14, 17 to 30, and 32, and Comparative Example 1, the test method 3-1 was performed. In Examples 15 and 16, the test method 3-2 was performed. In Example 31, the test method 3-3 was performed. In Comparative Example 2, the test method 2-1 in the contact step 2 was performed.

### (Test Method 3-1)

A vertically set column with a capacity of 300 ml was filled with 200 ml of a gel-type strongly acidic cation exchange resin (trade name “Amberlite IR-124Na”) that had been regenerated to H-type in advance using IN dilute hydrochloric acid. The cation exchange resin was thoroughly washed with 4000 g of pure water and then left to stand for 24 hours. Subsequently, the pure water injected into the column was kept at a constant temperature within the range of 15 to 25° C. Thereafter, the aqueous solution of the organic sulfonic acid compound adjusted to the same temperature as the pure water in the column was passed through the column at the space velocity (SV) listed in Table 1.

### (Test Method 3-2)

The aqueous solution of the organic sulfonic acid compound was passed through the column at the space velocity (SV) listed in Table 1 in the same manner as in the test method 3-1, except that the MR-type strongly acidic cation exchange resin (trade name “Amberlite 200CT”) was used instead of the gel-type strongly acidic cation exchange resin (trade name “Amberlite IR-124Na”) in the test method 3-1.

### (Test Method 3-3)

180 ml of the gel-type strongly acidic cation exchange resin (trade name “Amberlite IR-124Na”) in the test method 3-1 and 20 ml of an MR type weakly acidic cation exchange resin (trade name “Amberlite FPC3500”) were mixed uniformly. A vertically set column with a capacity of 300 ml was filled with the above mixed resin. Subsequently, the pure water injected into the column was kept at a constant temperature within the range of 15 to 25° C. Thereafter, the aqueous solution of the organic sulfonic acid compound adjusted to the same temperature as the pure water in the column was passed through the column at the space velocity (SV) listed in Table 1.

Chlorine contents, metal contents, and purification amounts per unit time of the aqueous solutions of the organic sulfonic acid compounds obtained in Examples 1 to 32 and Comparative Examples 1 to 5 were measured and evaluated by the following test methods. Table 2 shows the results.

### Chlorine Content

The chlorine ion content in the aqueous solution of the organic sulfonic acid compound after purification was determined by dropping a 0.01 mol/l silver nitrate aqueous solution using a potentiometric titrator (AT-610, Kyoto Electronics Manufacturing Co., Ltd.). The chlorine ion content in the aqueous solution of the organic sulfonic acid compound after purification was evaluated based on the following evaluation criteria.

### (Evaluation Criteria of Chlorine Ion (Aqueous Solution Equivalent Value))

A: Chlorine ion content is less than 20 ppm (excellent)
B: Chlorine ion content is 20 ppm or more and less than 50 ppm (good)
C: Chlorine ion content is 50 ppm or more and less than 100 ppm (fair)
D: Chlorine ion content is 100 ppm or more (unacceptable)

### Metal Content

Metal ion contents in the aqueous solution of the organic sulfonic acid compound after purification were measured using an inductively coupled plasma mass spectrometer (ICP-MS 7700, manufactured by Agilent Technologies). The metal ion contents in the aqueous solution of the organic sulfonic acid compound after purification were evaluated based on the following evaluation criteria.

### (Evaluation Criteria of Na Ion (Aqueous Solution Equivalent Value))

A: Na ion content is less than 30 ppb (excellent)
B: Na ion content is 30 ppb or more and less than 50 ppb (good)
C: Na ion content is 50 ppb or more and less than 80 ppb (fair)
D: Na ion content is 80 ppb or more (unacceptable)

### (Evaluation Criteria of K Ion (Aqueous Solution Equivalent Value))

A: K ion content is less than 5 ppb (excellent)
B: K ion content is 5 ppb or more and less than 20 ppb (good)
C: K ion content is 20 ppb or more and less than 50 ppb (fair)
D: K ion content is 50 ppb or more (unacceptable)

### (Evaluation Criteria of Ca Ion (Aqueous Solution Equivalent Value))

A: Ca ion content is less than 10 ppb (excellent)
B: Ca ion content is 10 ppb or more and less than 20 ppb (good)
C: Ca ion content is 20 ppb or more and less than 50 ppb (fair)
D: Ca ion content is 50 ppb or more (unacceptable)
   (Fe ion evaluation criteria (Aqueous Solution Equivalent Value))
A: Fe ion content is less than 10 ppb (excellent)
B: Fe ion content is 10 ppb or more and less than 20 ppb (good)
C: Fe ion content is 20 ppb or more and less than 50 ppb (fair)
D: Fe ion content is 50 ppb or more (unacceptable)

### Purification Amount per Unit Time

The purification amounts per hour were measured in terms of solid content for 100 ml of ion exchange resin in the contact step 2 and the contact step 3. The measured purification amount was evaluated based on the following evaluation criteria.
A: Purification amount is 10 g or more (excellent)
B: Purification amount is 5 g or more and less than 10 g (good)
C: Purification amount is less than 5 g (fair)

TABLE 2

| | Content | | | | | Purification amount per unit time (g) | |
|---|---|---|---|---|---|---|---|
| Category | Chlorine (ppm) | Na (ppb) | K (ppb) | Ca (ppb) | Fe (ppb) | Contact step 2 | Contact step 3 |
| Example 1 | A | A | A | A | A | A | A |
| Example 2 | A | A | A | A | A | A | A |
| Example 3 | A | A | A | A | A | A | A |
| Example 4 | A | A | A | A | A | A | A |
| Example 5 | A | A | A | A | A | A | A |
| Example 6 | A | A | A | A | A | A | A |
| Example 7 | A | A | A | A | A | A | A |
| Example 8 | A | A | A | A | A | A | A |
| Example 9 | A | A | A | A | A | A | A |
| Example 10 | A | A | A | A | A | A | A |
| Example 11 | A | A | A | A | A | A | A |
| Example 12 | A | A | A | A | A | A | A |
| Example 13 | A | A | A | A | A | A | A |
| Example 14 | A | A | A | A | A | A | A |
| Example 15 | A | A | A | A | A | A | A |
| Example 16 | A | A | A | A | A | A | A |
| Example 17 | A | A | A | A | A | A | B |
| Example 18 | A | A | A | A | A | B | A |
| Example 19 | A | A | A | A | A | A | B |
| Example 20 | A | A | A | A | A | C | C |
| Example 21 | A | B | B | B | B | A | A |
| Example 22 | A | C | C | C | C | A | A |
| Example 23 | B | A | A | A | A | A | A |
| Example 24 | B | A | A | A | A | A | A |
| Example 25 | B | A | A | A | A | A | A |
| Example 26 | B | B | B | B | B | A | A |
| Example 27 | C | A | A | A | A | A | A |
| Example 28 | C | C | C | C | C | A | A |
| Example 29 | C | A | A | A | A | A | A |
| Example 30 | A | A | A | A | A | A | A |
| Example 31 | A | A | A | A | A | A | A |
| Example 32 | C | C | C | C | C | A | A |
| Comparative | D | A | A | A | A | — | A |
| Example 1 | | | | | | | |
| Comparative | D | A | A | A | A | B | B |
| Example 2 | | | | | | | |
| Comparative | A | D | D | D | D | B | — |
| Example 3 | | | | | | | |
| Comparative | D | D | D | D | D | A | — |
| Example 4 | | | | | | | |
| Comparative | D | C | C | C | C | A | — |
| Example 5 | | | | | | | |

In Examples 1 to 32, the chlorine ion content in the aqueous solution of the purified organic sulfonic acid compound was reduced to less than 100 ppm. With regard to the metal contents, the Na ion content was reduced to less than 80 ppb. The K ion content, the Ca ion content, and the Fe ion content were reduced to less than 50 ppb. Furthermore, in Examples 1 to 19 and 21 to 32, the purification amount per unit time was 5 g or more in both the contact step 2 and the contact step 3. In Example 20, the solution concentration of the organic sulfonic acid compound contacting the exchange resin was low in both the contact step 2 and the contact step 3. Therefore, the purification amount per unit time did not exceed 5 g. However, evaluation results of the chlorine ion content and the metal contents were excellent. On the other hand, in Comparative Example 1, the contact step 2 was not performed. Therefore, the chlorine ion content was 100 ppm or more. In Comparative Example 2, the contact step 2 was performed after the contact step 3. Therefore, the chlorine ion content was 100 ppm or more. In Comparative Example 3, the contact step 3 was not performed. Therefore, the metal ion content was not sufficiently reduced. In Comparative Example 4, a mixed bed of the anion exchange resin and the cation exchange resin was used in the contact step 2. Therefore, neither the chlorine ion content nor the metal ion contents were sufficiently reduced. In Comparative Example 5, in the contact step 2, the column was filled with both the anion exchange resin and the cation exchange resin so that they were in contact with each other. That is, the anion exchange resin and the cation exchange resin were not separated from each other. Therefore, the chlorine ion content was not sufficiently reduced. With regard to the metal contents, the Na ion content was 50 ppb or more and less than 80 ppb. The K ion content, the Ca ion content, and the Fe ion content were 20 ppb or more and less than 50 ppb.

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. A method for producing an organic sulfonic acid compound, the method comprising a reaction step 1, a contact step 2, and a contact step 3, wherein
in the reaction step 1, an organic sulfonic acid compound is synthesized using a reaction base agent containing a chlorine-based compound,
in the contact step 2, a solution of the organic sulfonic acid compound obtained in the reaction step 1 is contacted with an anion exchange resin,
in the contact step 3, the solution of the organic sulfonic acid compound undergoing the contact step 2 is contacted with a cation exchange resin, and
the anion exchange resin in the contact step 2 and the cation exchange resin in the contact step 3 are separated from each other.

2. The method for producing the organic sulfonic acid compound, according to claim 1, wherein the organic sulfonic acid compound is a surfactant.

3. The method for producing the organic sulfonic acid compound, according to claim 1, wherein in the contact step 2, the solution having an organic sulfonic acid compound concentration of 1 to 50 mass % is contacted with the anion exchange resin.

4. The method for producing the organic sulfonic acid compound, according to claim 1, wherein in the contact step 3, the solution having an organic sulfonic acid compound concentration of 1 to 40 mass % is contacted with the cation exchange resin.

5. The method for producing the organic sulfonic acid compound, according to claim 1, wherein in the contact step 2, the solution is contacted with the anion exchange resin at a space velocity (an SV) of 0.3 to 4.9 h⁻¹.

6. The method for producing the organic sulfonic acid compound, according to claim 1, wherein in the contact step 3, the solution is contacted with the cation exchange resin at a space velocity (an SV) of 0.3 to 4.9 h⁻¹.
